(19) United States
(12) Patent Application Publication
(10) Pub. No.: US 2004/0146935 A1
(43) Pub. Date: Jul. 29, 2004

(54) Disease associated gene

(76) Inventors: Rosalinda Cusido Roberts, Columbia, MD (US); Jan Van Oostrum, Hohenweg (CH); Johannes Voshol, Rodersdorf (CH); Carol Ann Tamminga, Potomac, MD (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/471,567

(22) PCT Filed: Mar. 14, 2002
(86) PCT No.: PCT/EP02/02872

(30) Foreign Application Priority Data
Mar. 15, 2001 (US) 60276306

(51) Int. Cl. G01N 33/53; C07K 14/705

(52) U.S. Cl. 435/7.1; 530/358

(57) ABSTRACT

A method of sreening for compounds useful for the treatment of SSDs comprising contacting a test compound with a y-synuclein polypeptide and detecting interactions of the test compounds with said polypeptide or a nucleotide sequence encoding y-synuclein for the diagnosing of SSDs or susceptibility to SSDs, a kit useful for diagnosing SSDs or susceptibility to a SSDs, and a transgenic animal useful for the study of SSDs.

### FIELD OF THE INVENTION

The present invention relates to methods for diagnosing Schizophrenia Spectrum Disorders, as well as methods of screening for compounds capable of modulating said disorders. In particular, the invention provides γ-synuclein as a genetic determinant for schizophrenia and as a potential therapeutic target for the treatment of schizophrenia.

### BACKGROUND OF THE INVENTION

Schizophrenia Spectrum Disorders (SSDs) are a common mental illness affecting about 1% of the population. They are characterized by a constellation of psychotic symptoms, such as hallucinations, severely inappropriate emotional responses, disordered thinking and concentration, and erratic behavior and by social and occupational deterioration. Because SSDs cause a serious public health problem, there is an urgent medical need for search for novel drugs for the treatment of SSDs (Carpenter and Buchanan, N Engl J Med 330 (1994), 681-690).

Since the introduction of the polyvalent typical and atypical antipsychotic agents, there has been no breakthrough treatment for the disease for over thirty years. The identification of rational drug targets and, ideally, those that address the causes rather than the symptoms of the disorder, is severely hampered by (i) our low level of understanding of its molecular basis and (ii) the lack of animal models with both predictive and construct validity that can be used to assess the antischizophrenic potential of hypothetical drug targets. Without progress on both these fronts, truly efficacious treatments will come about only by virtue of serendipity.

The molecular and cellular biology of SSDs is poorly understood. Although genetic predisposition is an important factor in the etiology of SSDs, it is generally accepted that environmental factors are at least equally important. A recent Danish study (Mortensen et al., N Engl J Med 340 (1999), 603-608) demonstrated that, although a family history of SSDs is the single most important risk factor, on a population basis many more cases of SSDs are due to environmental risk factors. The observations that a variety of loci on different chromosomes appear to be related to SSDs (Riley and McGuffin, Am J Med Genet 97 (2000), 23-44), suggest that even the genetic component is very heterogeneous.

No SSDs susceptibility gene has been identified so far, so there is a need in the art for the identification of such genes. Identification of SSDs susceptibility genes would provide a fundamental understanding of the disease process from which a number of clinically important applications would arise. Susceptibility genes identified may lead to the development of therapeutics (such as, for instance, small molecule drugs, antisense molecules, antibody molecules) directly targeted to the gene or protein product of the gene, or may target the biochemical pathway of which the protein product is a part at an upstream or downstream location if the development of such drugs is easier than directly targeting the gene or its protein product.

The present invention provides for the first time a genetic component of SSDs and therefore makes available novel therapeutic and diagnostic methods.

### SUMMARY OF THE INVENTION

The present invention provides a polymorphism of γ-synuclein that makes individuals more susceptible to SSDs, thus providing for the first time a direct link between a gene and susceptibility to SSDs. As a result, the present invention is an important contribution to our understanding of the molecular basis of SSDs and could lead the way to novel pharmaceuticals and therapies for the treatment of SSDs.

Accordingly, the present invention provides, in one aspect, a method of screening for compounds useful for the treatment of SSDs comprising the steps of a) contacting a γ-synuclein polypeptide with the compounds to be screened and b) detecting interactions of said compounds with said polypeptide. In another aspect, the invention provides a method of screening for compounds capable of interfering with the onset of SSDs comprising the steps of a) contacting a γ-synuclein polypeptide with the compounds to be screened and b) detecting interactions of said compounds with said polypeptide. In a preferred embodiment, the present invention provides a γ-synuclein polypeptide with a mutation. The mutation is preferably at position 110 which is glutamic acid in the wild-type γ-synuclein. Most preferred is a substitution of glutamic acid by valine at position 110. The present invention also encompasses fragments of γ-synuclein, in particular fragments containing the glutamic to valine substitution at position 110.

In further aspect, the present invention provides the use of a γ-synuclein polypeptide, a fragment, variant or derivative thereof, for the screening of compounds useful for the treatment of SSDs. In a preferred embodiment, the γ-synuclein contains a mutation. In a more preferred embodiment the glutamic acid at position 110 is substituted by valine.

In another aspect, the invention provides a method of screening for agonist or antagonist compounds of γ-synuclein comprising the steps of a) incubating γ-synuclein expressing cells with a candidate compound, and b) assaying for the interference of said compounds with the interaction of γ-synuclein and other cellular proteins.

In another aspect, the invention provides a compound, identified by a method of screening for γ-synuclein, useful for the treatment of SSDs.

In a further aspect, the present invention provides a polymorphism or, more preferably, a single nucleotide polymorphism (SNP) of γ-synuclein involved in SSDs. The present invention also provides SNP probes useful for the study of SSDs or susceptibility to SSDs. In a preferred embodiment, the invention provides an oligonucleotide complementary to a part of the γ-synuclein coding sequence useful for the discrimination of a SNP at position 329 of the γ-synuclein coding sequence.

Another aspect of the invention relates to the use of a γ-synuclein polypeptide or a nucleotide sequence encoding γ-synuclein for the diagnosis of SSDs or susceptibility to SSDs.

In further aspect, the invention relates to a method for the diagnosis of SSDs or susceptibility to SSDs comprising the steps of a) obtaining cell(s) or a tissue sample from a potential patient, b) analyzing the nucleotide sequence of a gene or a part of a gene encoding γ-synuclein, and c) comparing the results obtained with a standard sequence. In a preferred embodiment, the invention provides a kit for the diagnosis of SSDs or susceptibility to SSDs according to this method.

In another aspect, the invention provides antibodies that specifically recognize an allelic form of the γ-synuclein that confers susceptibility to SSDs. In a preferred embodiment, the invention provides an antibody that binds to γ-synuclein polypeptides, or fragments thereof, said polypeptides having a mutation at amino acid position 110 where glutamic acid is substituted for valine, wherein said antibody will bind to an epitope comprising the mutation at position 110 and has a lower binding affinity for the corresponding non-mutated γ-synuclein or fragment thereof. Accordingly, in a preferred embodiment, the invention relates to a method for the diagnosis of SSDs or susceptibility to SSDs comprising the determination of the presence or absence of a glutamic acid at position 110 using a selective antibody. Envisaged is, for instance, a method for the diagnosis of SSDs or susceptibility to SSDs comprising the steps of a) obtaining cell(s) or a tissue sample from a potential patient, and b) determining the presence or absence of valine at position 110 of a γ-synuclein polypeptide with the selective antibody. In a preferred embodiment, the invention provides a kit for the diagnosis of SSDs or susceptibility to SSDs according to this method.

A further aspect of this invention is a kit useful for diagnosing SSDs or susceptibility to SSDs according to any claims 21 to 25 comprising at least one of the following components: a) a polynucleotide of the sequence of γ-synuclein, or a fragment thereof, encompassing a sequence containing a SNP, b) a nucleotide sequence complementary to that of (a), c) a γ-synuclein polypeptide or a fragment thereof, or d) an antibody to a γ-synuclein polypeptide or a fragment thereof, capable of detecting a mutation at position 110 of the polypeptide, the mutation preferably being a substitution of glutamic acid by valine.

Another aspect of the invention relates to transgenic animals. In one embodiment, the invention provides a transgenic animal, preferably a mammal, useful for the study of SSDs or susceptibility to SSDs having a completely or partially deleted γ-synuclein gene. In a further embodiment the invention provides a transgenic animal useful for the study of SSDs or susceptibility to SSDs comprising an exogenous human γ-synuclein gene. The endogenous γ-synuclein of such a transgenic animal can be completely or partially deleted. Another embodiment of the invention provides a transgenic animal useful for the study of SSDs, wherein the endogenous γ-synuclein is modified to include the polymorphism corresponding to position 110 in the human γ-synuclein polypeptide sequence.

### BRIEF DESCRIPTION OF THE DRAWINGS

Table I shows the distribution of γ-synuclein isoforms among all 40 individual anterior cingulate samples that were analyzed, in terms of the presence or absence of the Glu¹¹⁰ and Val¹¹⁰ variants. A shaded box indicates absence of the respective isoform.

FIG. 1 shows the mRNA sequence with GenBank accession number AF010126 (Ji et al., Cancer Res. 57 (1997), 759-764). The Val¹¹⁰ variant is deposited under AF 037207 (Ninkina et al., Hum. Mol. Genet. 7 (1998), 1417-1424).

FIG. 2 shows MALDI spectra of spots 278 and 438.

FIG. 3 shows the MSMS spectrum of peptides derived from Spot 438.

FIG. 4 shows the MSMS spectrum of peptides derived from Spot 278.

FIG. 5 shows the different phenotypes for γ-synuclein that were observed in the analysis of anterior cingulate samples by two-dimensional electrophoresis.

### DETAILED DESCRIPTION OF THE INVENTION

Any patents and publications cited herein reflect the knowledge in this field and are hereby incorporated by reference in entirety.

Terms used herein have the following meaning:

The term “Schizophrenia Spectrum Disorders” means a class of psychotic disorders, including, but not limited to, schizophrenia, schizophreniform disorder, schizoaffective disorder, paranoid disorder, schizoid disorder, schizotypal disorder and delusional disorder. The diagnostic criteria are described in the Diagnostic and Statistical Manual of Mental Disorders (fourth edition 1996, “DSM-IV”).

The term “polypeptide” is used interchangeably herein with the terms “polypeptides” and “protein(s)”. “γ-Synuclein polypeptide” refers to a polypeptide having the same amino acid sequence as γ-synuclein derived from nature. Such native sequences can be isolated from nature or can be produced by recombinant and/or synthetic means. Naturally occurring truncated or naturally occurring variant forms (e.g., alternatively spliced forms) and naturally occurring allelic variants of γ-synuclein are encompassed. “Wild-type” refers to the allelic form of a gene that is considered as the original form of the gene, i.e. before a mutation occurred. Wild-type γ-synuclein means the γ-synuclein amino acid sequence corresponding to the coding sequence as deposited in Genbank under accession number AF010126 (Ji et al., Cancer Res. 57 (1997), 759-764). In the present patent, the numbering of nucleotides of the γ-synuclein gene or of amino acids of the γ-synuclein polypeptide refers to the numbering as disclosed in Genbank, accession number AF010126.

The term “antagonist” is used in the broadest sense, and includes any molecule that partially or fully blocks, inhibits, or neutralizes a biological activity of native γ-synuclein. In a similar manner, the term “agonist” is used in the broadest sense and includes any molecule that mimics a biological activity of a native γ-synuclein polypeptide. Suitable agonist or antagonist molecules specifically include agonist or antagonist antibodies or antibody fragments, fragments or amino acid sequence variants of native γ-synuclein polypeptides, peptides, antisense molecules, and small organic molecules.

“Modulate” means a partial or complete inhibition, or an increase, in the activity of a polypeptide.

The term “antibody” is used in the broadest sense and specifically covers, for example, single anti-γ-synuclein monoclonal antibodies (including agonist, antagonist, and neutralizing antibodies), anti-γ-synuclein antibody compositions with polyepitopic specificity, single chain anti-γ-synuclein antibodies and fragments of anti-l-synuclein antibodies. The term “monoclonal antibody” as used herein refers to an antibody obtained from a population of substantially homogeneous antibodies, i.e. the individual antibodies comprising the population are identical except for possible naturally-occurring mutations that can be present in minor amounts.

A “small organic molecule” is defined herein to have a molecular weight below about 500 Daltons.

In a pharmacological sense, in the context of the present invention, a “therapeutically effective amount” of an active agent such as a γ-synuclein polypeptide or agonist or antagonist thereto or an anti-γ-synuclein antibody, refers to an amount effective in the treatment of SSDs in a mammal and can be determined empirically.

“Isolated” refers to material removed from its original environment.

“Hybridization” or “hybridizes” refers to any process by which a strand of a polynucleotide binds with a complementary strand through base pairing.

“Stringent conditions” refer to experimental conditions which allow up to 20% base pair mismatches, typically two 15 minute washes in 0.1×SSC (15 mM NaCl, 1.5 mM sodium citrate, pH 7.0) at 65° C.

“Homology” or “homologous” refers to a degree of similarity between nucleotide or amino acid sequences, which may be partial or, when sequences are identical, complete.

“Antisense” refers to selective inhibition of protein synthesis through hybridization of an oligo- or polynucleotide to its complementary sequence in messenger RNA (mRNA) of the target protein. The antisense concept was first proposed by Zamecnik and Stephenson, Proc. Natl. Acad. Sci. USA 75 (1978): 280-284; Proc. Natl. Acad. Sci. USA 75 (1978): 285-288) and has subsequently found broad application both as an experimental tool and as a means of generating putative therapeutic molecules (Alama, A. et al., Pharmacol. Res. 36 (1997):171-178; Dean, N. M. et al., Biochem. Soc. Trans. 24 (1996):623-629; Bennet, C. F., J. Pharmacol. Exp. Ther. 280:988-1000; Crooke, S. T., Antisense Research and Applications, Springer).

The term “variant” as used herein means, in relation to amino acid sequences, an amino acid sequence that is altered by one or more amino acids. The changes may involve amino acid substitution, deletion or insertion. In relation to nucleotide sequences, the term “variant” as used herein means a nucleotide sequence that is altered by one or more nucleotides; the changes may involve nucleotide substitution, deletion or insertion.

As used herein, a “derivative” of a polypeptide of the invention is a polypeptide of the invention that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th ed., Mack Publishing Co., Easton, Pa. (1980).

The present invention relates to a genetic component in Schizophrenia Spectrum Disorders. Therefore, the invention could be very useful for the development of new drugs or medicaments for the treatment of SSDs, because so far, our poor understanding of the molecular and cellular biology of SSDs has severely hampered the identification of rational drug targets and the development of new anti-psychotic drugs. This invention now provides a genetic link between I-synuclein and susceptibility to Schizophrenia Spectrum Disorders.

γ-Synuclein is a member of a protein family termed synucleins. The other members identified so far are α-synuclein and β-synuclein (reviewed in Clayton and George, Trends Neurosci 21 (1998), 249-254). The synucleins are a family of small soluble proteins expressed in the nervous system and localized in synaptic terminals. The three proteins, although homologous, are encoded by three different genes mapped to different chromosomes (Campion, D. et al. Genomics 26 (1995):254-257; Chen, X. et al. 1995 Genomics 26:425427, Spillantini, M. G. et al. Genomics 27 (1995):379-381, Lavedan, C. et al. Hum. Genet. 103 (1998):106-112). The functions of synucleins still remain elusive. α- and β-synuclein have been shown to bind and stimulate aggregation of β-amyloid peptide making them candidates genes for being risk factors for Alzheimer's Disease (Jensen, P. H. et al. Biochem. J. 323 (1997): 539-546; Yoshimoto, M. et al. Proc. Natl. Acad. Sci., 92 (1995): 9141-9145). Additionally, α-synuclein was shown to harbor an alanine to threonine substitution at position 53 in four pedigrees with autosomal-dominantly inherited familial Parkinson's disease (Polymeropoulos, M. H. et al. Science 276 (1997): 2045-2047). Studies have also shown that α-synuclein is a component of Lewy bodies in sporadic Lewy body diseases including Parkinson's disease and dementia with Lewy bodies (Spillantini, M. G. et al. Nature 388 (1997): 839-840; Takeda, A. et al. Am. J. Pathol. 152 (1998):367-372).

An attempt to identify an association of γ-synuclein and the risk for a mental illness failed (Luedecking, E. K. et al. Neuroscience Letters 261 (1999): 186-188). However, γ-Synuclein, also known as breast cancer specific gene 1, BCSG1, was shown to be highly expressed in a breast cancer cDNA library but scarcely in a normal breast cDNA library (Ji, H. et al. Cancer Res. 57 (1997): 759-764).

The present invention provides a new function for γ-synuclein as a factor of susceptibility to SSDs and discloses a mutation that promotes the onset of SSDs. This makes γ-synuclein polypeptides an attractive target for the screening of compounds capable of interfering with the onset of SSDs or useful for the treatment of SSDs. Such compounds could lead to the development of drugs preventing the onset of SSDs or alleviate the symptoms of SSDs.

Accordingly, the present invention provides a method of screening for compounds capable of interfering with the onset of SSDs and a method of screening for compounds useful for the treatment of SSDs. The screening assays for such compounds can be designed in a variety of ways and are well known to a person skilled in the art. Some examples of such screening methods are given below, but the invention could also be practiced with other screening method known a person of skill in the art. The term compound is to be understood in its broadest sense and includes, but is not limited to polypeptides, antibodies, small organic molecules from natural sources or chemically synthesized.

This invention is particularly useful for screening compounds using γ-synuclein polypeptides or fragments thereof in any of a variety of drug screening techniques. In a preferred embodiment of the invention γ-synuclein polypeptides or fragments thereof contain a mutation at position 10, preferably a substitution of glutamic acid 110 to valine. The γ-synuclein polypeptide or fragment employed in such a test can either be free in solution, affixed to a solid support, borne on a cell surface, or located intracellularly.

One method of drug screening is, for instance, binding assays. In binding assays, the interaction is binding and the complex formed can be isolated or detected in the reaction mixture. In a particular embodiment, the γ-synuclein polypeptide or fragment thereof or the drug candidate is immobilized on a solid phase, e.g. on a microtiter plate, by covalent or non-covalent attachments. An immobilized antibody, e.g. a monoclonal antibody, specific for γ-synuclein to be immobilized can be used to anchor it to a solid surface. The assay is performed by adding the non-immobilized component, which may be labeled by a detectable label, to the immobilized component, e.g. the coated surface containing the anchored component. When the reaction is complete, the non-reacted components are removed, e.g. by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, be using a labeled antibody.

Another method of drug screening utilizes eukaryotic or prokaryotic host cell which are stably transformed with recombinant nucleic acids expressing the γ-synuclein polypeptide or a fragment thereof. Drugs can be screened against such transformed cells in competitive binding assays. Such cells, either in viable or fixed from, can be used for standard binging assays. One can measure, for example, the formation of complexes between γ-synuclein or a fragment thereof and the agent being tested. Alternatively, one can examine the diminution in complex formation between the γ-synuclein and its target cells or target receptors caused by the agent being tested. Thus, the present invention provides methods of screening for drugs or any other agents which can affect γ-synuclein associated SSDs. These methods comprise contacting such an agent with a γ-synuclein polypeptide or fragment thereof and assaying (i) for the presence of a complex between the agent and the γ-synuclein polypeptide or fragment thereof, or (ii) for the presence of a complex between the γ-synuclein polypeptide or fragment thereof and the cell, by methods well known in the art. In such competitive binding assays, the γ-synuclein polypeptide or fragment thereof is typically labeled. After suitable incubation, free or uncomplexed label is a measure of the ability of the particular agent to bind to γ-synuclein or to interfere with the γ-synuclein polypeptide/cell complex.

Another technique for drug screening provides high throughput screening for compounds having suitable binding affinity to a polypeptide as described in WO 84/03564, published on Sep. 13, 1984. Briefly stated, large numbers of different small peptide test compounds are synthesized on a solid substrate, such as plastic pins or some other surface. As applied to a γ-synuclein polypeptide, the peptide test compounds are reacted with γ-synuclein polypeptide and washed. Bound γ-synuclein polypeptide is detected by methods well known in the art. Purified γ-synuclein polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding γ-synuclein polypeptide specifically compete with a test compound for binding to γ-synuclein polypeptides or fragments thereof. In this manner, the antibody can be used to detect the presence of any peptide which shares one or more antigenic determinants with γ-synuclein polypeptide.

The present invention further encompasses methods of screening compounds to identify those that mimic γ-synuclein polypeptide (agonists) or prevent the effect of the γ-synuclein (antagonists). Screening assays for antagonist drug candidates are designed to identify compounds that bind or complex with the γ-synuclein polypeptide or otherwise interfere with the interaction of γ-synuclein with other cellular proteins. Such screening assays will include assays amenable to interaction of the high-throughput screening of chemical libraries, making them particularly suitable for identifying small molecule drug candidates. The assays can be performed in a variety of formats, including protein-protein binding assays, immunoassays, and cell-based assays, which are well characterized in the art.

This invention can also be practiced by assaying for proteins interacting with γ-synuclein polypeptides using methods for the detection of protein-protein interactions. Such assays include traditional approaches, such as, e.g. crosslinking, co-immunoprecipitation, and co-purification through gradients or chromatographic columns. In addition, protein-protein interactions can be monitored by using a yeast-based genetic system (Fields et al., Nature 340 (1989): 245-246; Chien et al., Proc. Natl. Acad. Sci. USA 88 (1991): 9578-9582; Chevray et al., Proc. Natl. Acad. Sci. USA 89 (1991): 5789-5793.

A preferred embodiment of the present invention is related to an isoform of γ-synuclein that is caused by an A/T single nucleotide polymorphism (SNP) at position 329 of the γ-synuclein coding sequence. This SNP causes a glutamic acid to valine change at position 110 of γ-synuclein (Glu 110 Val).

Accordingly, the invention provides SNP probes which are useful in classifying people according to their types of genetic variation. The SNP probes according to the invention are oligonucleotides which can discriminate between alleles of a SNP nucleic acid in conventional allelic discrimination assays. As used herein, a “SNP nucleic acid” is a nucleic acid sequence which comprises a nucleotide which is variable within an otherwise identical nucleotide sequence between individuals or groups of individuals, thus existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning.

The SNP probes according to the invention are oligonucleotides that are complementary to a SNP nucleic acid. The term “complementary” means exactly complementary throughout the length of the oligonucleotide in the Watson and Crick sense of the word. In certain preferred embodiments, the oligonucleotides according to this aspect of the invention are complementary to one allele of the SNP nucleic acid, but not to any other allele of the SNP nucleic acid. In a preferred embodiment of the invention an oligonucleotide encompassing the SNP at position 329 of the coding sequence corresponding to γ-synuclein is used. This oligonucleotide can be complementary to the wild-type γ-synuclein sequence or to any allelic form of it. Oligonucleotides according to this embodiment of the invention can discriminate between alleles of the SNP nucleic acid in various ways. For example, under stringent hybridization conditions, an oligonucleotide of appropriate length will hybridize to one allele of the SNP nucleic acid, but not to any other allele of the SNP nucleic acid. Preferably, the oligonucleotide is labeled, most preferably by a radiolabel or a fluorescent label. Alternatively, an oligonucleotide of appropriate length can be used as a primer for PCR, wherein the 3′ terminal nucleotide is complementary to one allele of the SNP nucleic acid, but not to any other allele. In this embodiment, the presence or absence of amplification by PCR determines the haplotype of the SNP nucleic acid.

The present invention also provides methods of diagnosing SSDs or susceptibility to SSDs. Detection of a mutated form of a γ-synuclein polypeptide will allow a diagnosis of SSDs or susceptibility to SSDs, since the invention provides the link between γ-synuclein and SSDs. Individuals carrying mutations in the γ-synuclein gene may be detected at the DNA level by a variety of techniques. Nucleic acids for diagnosis may be obtained from a patient's cell(s), such as from blood, urine, saliva, tissue biopsy, and autopsy material. The genomic DNA may be used directly for detection or may be amplified by a polymerase chain reaction (PCR) or other amplification techniques prior to analysis. RNA or cDNA may also be used for the same purpose. Point mutations can be identified by hybridizing amplified DNA to radiolabeled RNA encoding γ-synuclein polypeptide, or alternatively, radiolabeled antisense DNA sequences encoding the γ-synuclein polypeptide. Perfectly matched sequences can be distinguished from mismatched duplexes, for example by RNase A digestion or by differences in melting temperatures. DNA sequence differences may also be detected by alterations in electrophoretic mobility of DNA fragments in gels, with or without denaturing agents, or by direct DNA sequencing (e.g., Myers et al., Science (1985) 230:1242). Preferred methods to detect a mutant sequence include without limitation Single-Strand Conformation Polymorphism technique (SSCP), Denaturing Gradient Gel Electrophoresis (DGGE), conformation sensitive gel electrophoresis (CSGE) or the FAMA technique described in PCT application WO 95/07361. Sequence changes at specific locations may also be revealed by nuclease protection assays, such as RNase and S1 protection or the chemical cleavage method (see Cotton et al., Proc Natl Acad Sci USA (1985) 85: 4397-4401. Thus, the diagnostic assays offer a process for diagnosing or determining a susceptibility to SSDs through detection of mutation in the γ-synuclein gene by the methods described.

In a preferred aspect, the invention provides a method of diagnosing predisposition of a subject to SSDs comprising determining the presence or absence of a sequence polymorphism in a γ-synuclein polynucleotide, preferably at a position of the DNA of the subject that corresponds to position 329 of the coding sequence for γ-synuclein. Such sequence polymorphisms correlating with SSDs may preferably alter the amino acid sequence in the encoded polypeptide, but may additionally or exclusively affect expression levels of the polypeptide or the way in which a transcript is processed.

Another preferred aspect of this invention is a method of detecting genetic abnormality in a subject which comprises incubating a genetic sample from the subject with a γ-synuclein polynucleotide probe, under conditions where the probe hybridizes to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to developing SSDs. Another aspect of this invention is a method of detecting a polymorphism, preferably at a position that corresponds to position 329 of the coding sequence for γ-synuclein, in the nucleotide sequence of a γ-synuclein in a patient which comprises amplifying a target nucleotide sequence in DNA from the patient by PCR using a suitable pair of primers which target the sequence to be amplified and analyzing the amplified sequence to determine any polymorphism present therein.

In another embodiment, the method comprises measuring at least one activity of γ-synuclein. For example, regulation of the expression of a gene by a γ-synuclein can be determined. Comparison of the results obtained with results from similar analysis performed on γ-synuclein proteins from healthy subjects is indicative of whether a subject has an abnormal γ-synuclein activity. In one embodiment, the method comprises determining whether a subject has an abnormal mRNA and/or protein level of γ-synuclein, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the γ-synuclein protein or mRNA level is determined and compared to the level of γ-synuclein protein or mRNA level in a healthy subject. An abnormal level of γ-synuclein polypeptide or mRNA level is likely to be indicative of an aberrant γ-synuclein activity.

Another aspect of the invention is a method of diagnosing SSDs by detecting polymorphism in a subject by treating a tissue sample from the subject with an antibody to a polymorphic variant of a γ-synuclein and detecting binding of said antibody. A preferred embodiment of the present invention is a polymorphic variant at position 110 of γ-synuclein, most preferably a Glu 110 Val mutation. A person of skill in the art would know how to produce such an antibody (see, for instance, Harlow, E. and Lane, eds., 1988, “Antibodies: A Laboratory Manual”, Cold Spring Harbor Press, Cold Spring Harbor). Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The invention also includes kits for the detection of an abnormality in the polynucleotide sequence of an individual's γ-synuclein gene. Hybridization kits for such detection comprise a probe of the invention as hereinbefore described, which probe may be modified by incorporation of a detectable, e.g. chemiluminescent or fluorescent, label therein, and may include other reagents such as labeling reagents, i.e. reagents to incorporate a detectable label such as a radioactive isotope, chemiluminescent or fluorescent group into a hybridized product, and buffers. PCR amplification kits comprise primer pairs such as those described above together with a DNA polymerase such as Taq polymerase, and may include additional reagents, such as an amplification buffer and the like. Specific embodiments of the PCR amplification kits can include additional reagents specific for a number of techniques that detect polynucleotide changes, including CSGE and DNA sequencing.

Thus in another aspect, the present invention relates to a diagnostic kit which comprises at least one of the following components:

(a) a polynucleotide of the sequence of γ-synuclein, or a fragment thereof, encompassing a sequence containing an SNP, preferably at position 329 of the coding sequence for γ-synuclein;

(b) a nucleotide sequence complementary to that of (a);

(c) a γ-synuclein polypeptide or a fragment thereof; or

(d) an antibody to a γ-synuclein polypeptide or a fragment thereof, capable of detecting a mutation at position 110 of the polypeptide, preferable a substitution of glutamic acid by valine.

Such a kit will be of use in diagnosing SSDs or susceptibility to SSDs.

The present invention also provides an animal model to study SSDs and susceptibility to SSDs. Such studies can be performed using transgenic animals. For example, one can produce transgenic mice, which contain a specific allelic variant of a γ-synuclein gene. These mice can be created, e.g., by replacing their wild-type γ-synuclein gene with an allele of the human γ-synuclein gene. For instance, the human γ-synuclein allele with the Glu 110 Val mutation could be introduced into such mice. The response of these mice to specific γ-synuclein therapeutics can then be determined.

In a preferred embodiment, the present invention provides a transgenic mammalian animal, said animal having cells incorporating a recombinant expression system adapted to express γ-synuclein (preferably human γ-synuclein, more preferably human γ-synuclein containing the Glu 110 Val polymorphism). Generally, the recombinant expression system will be stably integrated into the genome of the transgenic animal and will thus be heritable so that the offspring of such a transgenic animal may themselves contain the transgene. Transgenic animals can be engineered by introducing the coding portion of the γ-synuclein gene into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g. baboons, chimpanzees and monkeys. Techniques known in the art to introduce a transgene into such animals include pronucleic microinjection (U.S. Pat. No. 4,873,191); retrovirus-mediated gene transfer into germ lines (e.g. Van der Putten et al. 1985, Proc. Natl. Acad. Sci. USA 82: 6148-6152); gene targeting in embryonic stem cells (Thompson et al., Cell 56 (1989), 313-321); electroporation of embryos and sperm-mediated gene transfer (for a review, see for example, U.S. Pat. No. 4,736,866). For the purpose of the present invention, transgenic animals include those that carry the transgene only in part of their cells (“mosaic animals”). The transgene can be integrated either as a single transgene, or in concatamers. Selective introduction of a transgene into a particular cell type is also possible by following, for example, the technique of Lasko et al., Proc. Natl. Acad. Sci. USA 89 (1992): 6232-6236. Particular cells could also be targeted for γ-synuclein incorporation with tissue-specific enhancers. The expression of the transgene can be monitored by standard techniques such as in situ hybridisation, Northern Blot analysis, PCR or immunocytochemistry. Transgenic animals that include a copy of a transgene encoding γ-synuclein introduced into the germ line of the animal at an embryonic stage can be used to examine the effect of increased expression of DNA encoding γ-synuclein. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human bomologues of γ-synuclein can be used to construct a γ-synuclein “knock out” animal which has a defective or altered gene encoding γ-synuclein as a result of homologous recombination between the endogenous gene encoding γ-synuclein and altered genomic DNA encoding γ-synuclein introduced into an embryonic stem cell of the animal. A portion of the genomic DNA encoding γ-synuclein can be deleted or replaced with another gene, such as a gene encoding a selectable marker which can be used to monitor integration. Typically, several kilobases of unaltered flanking DNA (both at the 5′ and 3′ ends) are included in the vector (see e.g. Thomas et al., Cell 51 (1987): 503-512 for a description of homologous recombination vectors). The vector is introduced into an embryonic stem cell line (e.g. by electroporation) and cells in which the introduced DNA has homologously recombined with the endogenous DNA are selected (see e.g. Li et al., Cell 69 (1992): 915-926). The selected cells are then injected into a blastocyst of an animal (e.g. mouse or rat) to form aggregation chimeras (see e.g. Bradley, in Teratocarcinomas and Embryonic Stem Cells: A Practical Approach, E. J. Robertson, ed., IRL, Oxford 1987, pp.113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized, for instance, for their ability to defend against certain pathological conditions and for their development of pathological conditions due to absence of the γ-synuclein polypeptide.

In practicing the present invention, many conventional techniques in molecular biology, microbiology, and recombinant DNA are used. These techniques are well known and are explained in, for example, Current Protocols in Molecular Biology, Volumes I, II, and m, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The following Examples illustrate the present invention, without in any way limiting the scope thereof.

### EXAMPLES

### Example 1

Sample Preparation

Brain samples were obtained from Maryland Brain Collection, associated with the Maryland Psychiatric Research Center (MPRC). This collection currently holds over sixty brains from schizophrenic patients, some of which come from patients known to the MPRC for some time before death. A similar number of “normal” individuals is also available. MPRC operates a policy of extremely careful and verified diagnosis together with high quality control on the storage of the tissue. The dissection of areas of interest is done with high consistency. A critical point is the choice of brain regions to be analyzed. For schizophrenics with predominantly positive symptoms, such as hallucinations and delusions, the anterior cingulate cortex and the hippocampus are the two brain regions, most likely to exhibit alterations. This is based on the extensive database of the MPRC on the neurocircuitry underlying these facets of the disease (Tamminga et. al., Arch. Gen. Psych. 49:522-530, 1992; Buchanan et. al., 155 (1998):1049-1055).

For this study, a total of 40 brain samples derived from the anterior cingulate cortex was used: 20 brain samples of patients diagnosed with schizophrenia with predominantly positive symptoms and 20 brain samples of matched control samples. The diagnosis of schizophrenia was confirmed retrospectively by two independent psychiatrists, according to DSM-IV criteria. Samples were identified by numbers from 1-40 and processed as much as possible in pairs of one sample from the diseased and one from the control group. After all samples had been processed and run on two-dimensional gels, the assignment of the 40 samples to their respective groups was revealed to facilitate analysis. The identity of the groups was not revealed at that stage.

The tissue samples were processed the following way: Tissue samples (0.5-1 g) were homogenized in lysis buffer, 7 M urea, 2 M thiourea, 4% CHAPS, 1% DTT with 3 μg/ml DNAse I (Sigma D-4527) and 1.5 μg/ml RNAse A (Sigma R-5500), in a Potter-Elvehjem type glass homogenizer on ice. Samples were homogenized with 8 strokes at 800 rpm, kept at room temperature for 15 min and treated once more with three strokes at 800 rpm. Subsequently, tissue homogenates were centrifuged at 100'000 g, and the supernatants frozen at −80 ° C. before performing two-dimensional electrophoresis as described below. Pellets were stored separately.

### Example 2

Differential Analysis of Protein Expression in Schizophrenia Patients with Predominantly Positive Symptoms:

Two-dimensional electrophoresis was carried out using a modification of the method of Bjellqvist et al. (Bjellqvist B et al. (1993) Electrophoresis 14, 1357-1365). Three (five in case of the pooled samples) gels were run and stained in parallel for each sample. For the first dimension, samples of approximately 1.0 mg protein were loaded onto pH 4.5-5.5 linear IPG strips (Pharmacia) by reswelling the strips in sample solution (Sanchez J-C. et al. (1997) Electrophoresis 18, 324-327). Iso-electric focusing was performed on the Multiphor II apparatus (Pharmacia) for approximately 80 kVh at 20° C., essentially using the voltage gradient recommended by the manufacturer. After focusing, IPG strips were equilibrated as described in Bjellqvist B. et al. (1993), Electrophoresis 14, 1357-1365) with 2% DTT in the first step and 5% iodoacetamide in the second step. For the second dimension, IPG strips were applied to 20×25 cm (IsoDalt, Hoefer) SDS-PAGE gels (12% T, 2.6% C), which were run overnight at 100 V and 15° C. Gels were either fixed in 40% ethanol/10% acetic acid and stained overnight with Sypro Ruby (Molecular Probes) or with colloidal Coomassie Blue G-250, using the method of Anderson (Anderson L. (1991) Two-dimensional electrophoresis, 2nd ed., Large Scale Biology Press, Rockville Md.). Stained gels were digitized with the FLA-3000 fluorescence imager (Fuji) as TIFF files with 16-bit pixel depth. Before starting image analysis with the Melanie 3 program (Genebio), image size was reduced by a factor of 2 in both directions, by averaging neighboring pixels, to reduce noise. Spot tables and matching data (pairs reports) were exported from Melanie for statistical analysis with the Impressionist software package (GeneData). Since the digitized images sometimes contain artefacts, due to incomplete resolution of spots or background staining, differences were always checked and corrected, if necessary, by visual analysis of the original gels.

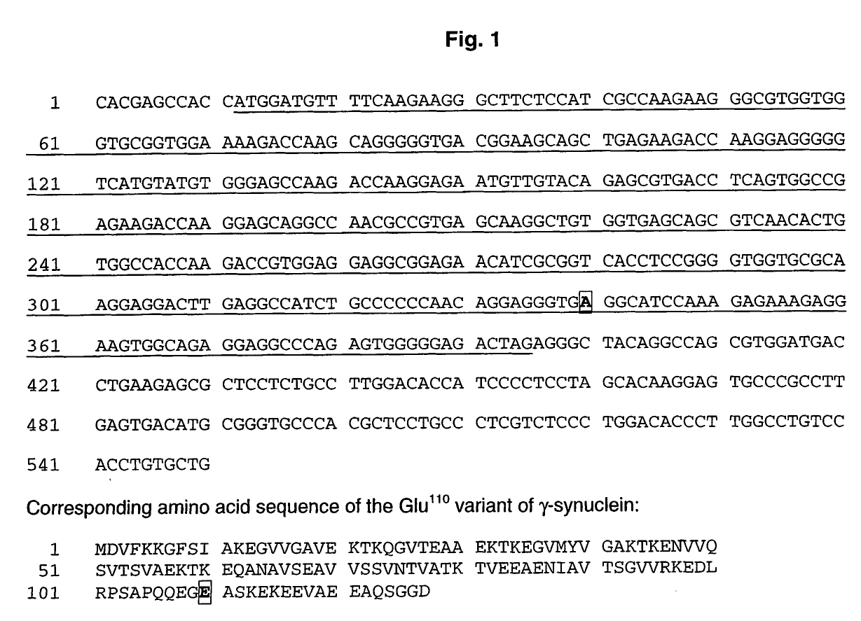

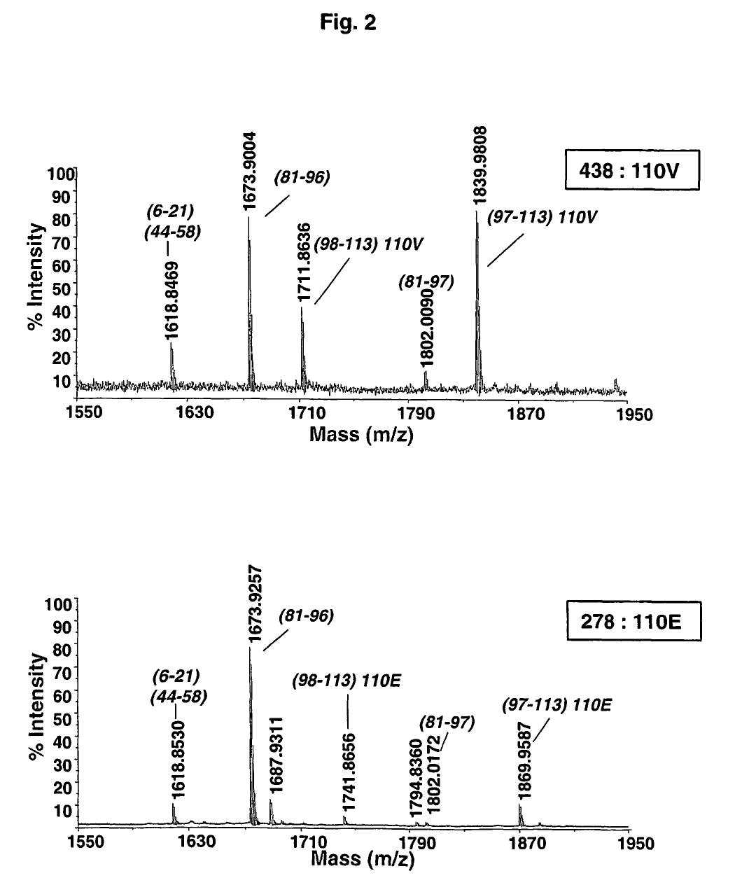

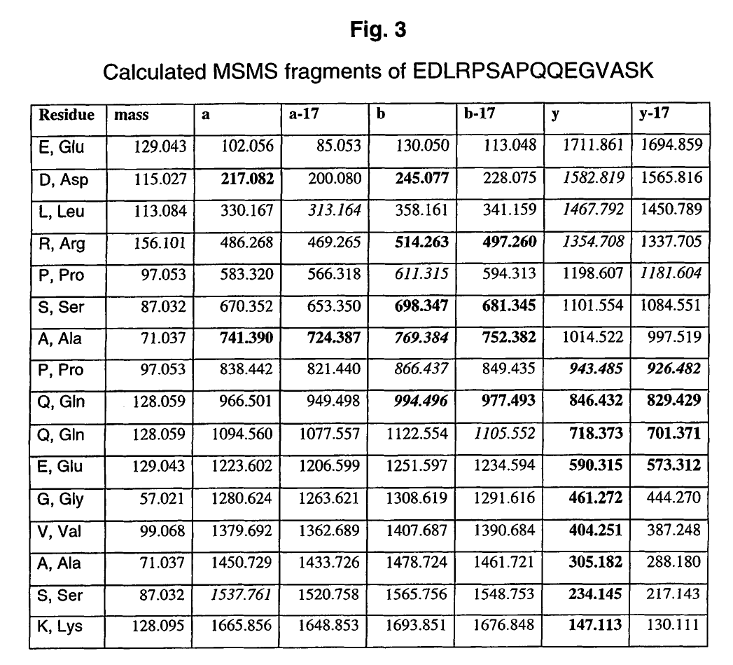

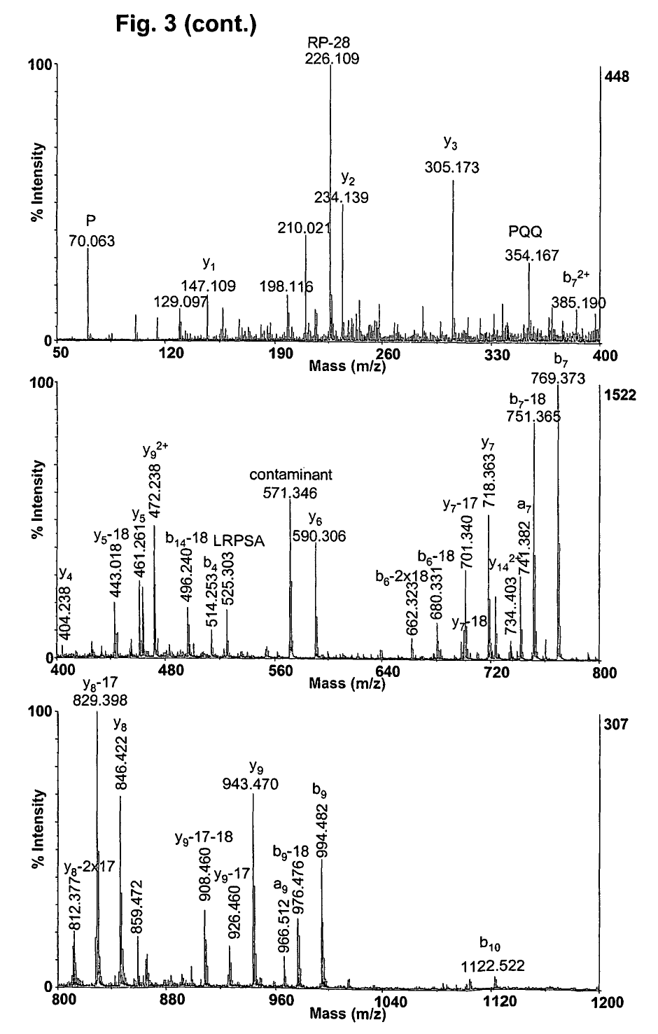

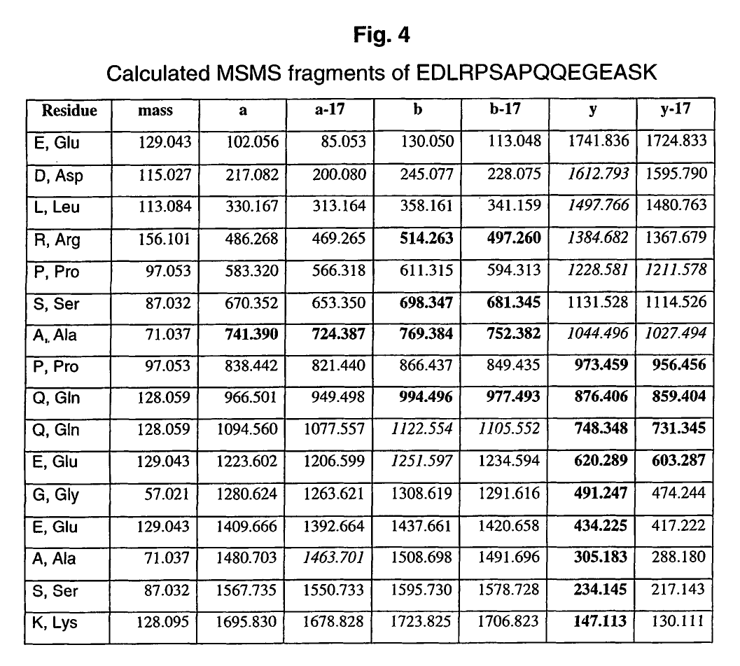

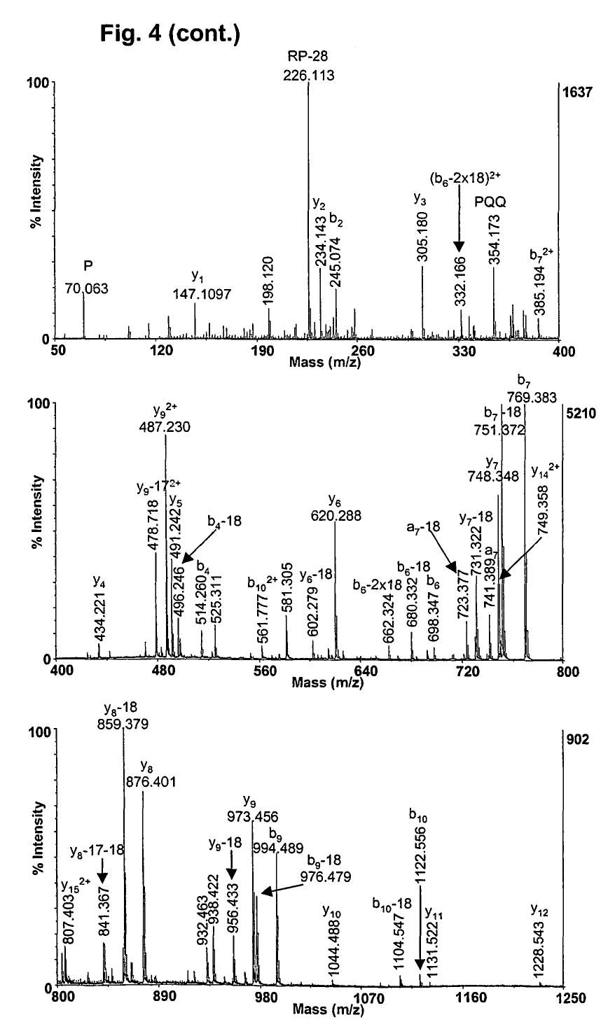

Differential analysis of protein expression between patients and controls was first performed on the anterior cingulate cortex, one of the brain regions thought to play in important role in positive symptoms. The analysis is carried out pH gradient by pH gradient with 120 gels per experiment (40 samples in triplicate). The data described below are derived from the pH range 4.5-5.5. Around 30 spots showed high variability between individuals, including a few with a more or less pronounced association with one of the groups. To increase the confidence in the spots that seemed to differ between groups, it was decided to run a pooled comparison in addition to the analysis of all individual samples. For this purpose the samples were combined into two pools of 10 for each group. The analysis of the pools confirmed the most consistent difference to be in the level of protein spot 438, which was downregulated in group 2 compared to group 1. This differences was statistically significant (T-test), group 1 vs. group 2: p<0.01. This protein spot was readily identified by MALDI mass spectrometry (see Example 3 and FIG. 2) as γ-synuclein (SwissProt accession nr. O76070). Detailed analysis by MALDI and nanospray-ESMSMS of spot 438 and a neighbouring spot 278 (Example 3, FIGS. 3 and 4) showed that two major isoforms of γ-synuclein are found in the anterior cingulate, with a known SNP, an A to T point mutation (FIG. 1, boxed residue), being the difference between them (Ninkina et al., Hum. Mol. Genet. 7 (1998), 1417-1424; Luedecking et al., 1999, Neurosci Lett 261:186-188.). This SNP causes a Glu to Val change at position 110 of the protein and a concomitant change in charge, which makes it detectable in two-dimensional electrophoresis as two spots with the same apparent molecular weight, but with different isoelectric points. Spot 438 represents the 110V isoform, whereas spot 278 has a Glu at position 110 (FIGS. 2-4).

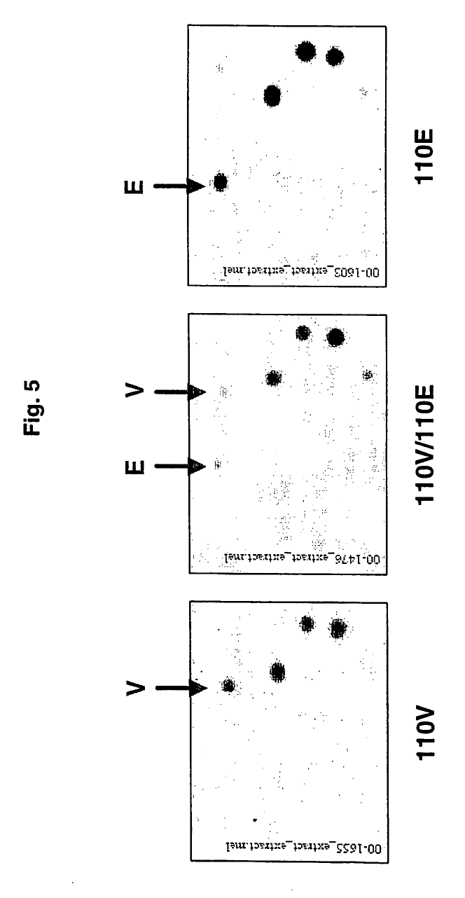

As expected, analysis of the individual samples revealed three possible phenotypes: the homozygous for either 110E or 110V and the heterozygous that has both (FIG. 5). The differential expression is explained by the presence of the 110V in only 4 of the 20 individuals in group 2, whereas it is present in 13 of 20 members of group 1, two of which are homozygous for 110V (Table II). The derived allele frequencies for the 110V and 110E allele are 0.375 (15/40): 0.625 (25/40) in group 1 and 0.100 (4/40): 0.900 (36/40) in group 2. These ratios are significantly different at the p<0.005 level (Pearson's chi-square test).

Following the analysis group 1, in which a high number of individuals express the 110V isoform, was identified as the patient group. Therefore the data presented here clearly suggest that this isoform of γ-synuclein is associated with schizophrenia. Since the underlying alteration is a genetic polymorphism, it is likely to be one of the genetic risk factors for a predisposition to the disease, since this protein is expressed in the brain (Lavedan et al., 1998, Hum Genet 103:106-112) and is thought to be involved in synaptic function and plasticity (Clayton and George, Trends Neurosci 21 (1998):249-254).

### Example 3

Identification of Gel Spots of Interest:

Protein spots of interest on the 2D-gel were analyzed the following way: Protein spots of interest were excised, transferred to 96-well plates and subjected to in-gel trypsin digestion, essentially according to Shevchenko A. et al. (1996) Anal. Chem. 66, 850-858. After reduction, alkylation and overnight digestion with trypsin, peptides were finally eluted with 5% formic acid.

The peptides were further analyzed by MALDI Mass Spectrometry: a-cyano4-hydroxy-cinnamic acid/nitrocellulose matrices were prepared by a modified version of the fast evaporation technique of Jensen O. N. et al. (1996) Rapid Comm. Mass Spectrom. 10, 1371-1378. A solution of nitrocellulose (Trans-Blot Transfer Medium, Bio-Rad, Glattbrugg, Switzerland) in acetone (10 mg in 0.5 mL) was admixed to a suspension of a-cyano-4-hydroxy-cinnamic acid (Sigma, Buchs, Switzerland) in isopropanol (20 mg in 0.5 mL,). 0.5 mL of this solution was applied to the sample plate followed by 0.8 mL of sample solution in 15 % CH3CN/H2O/2% HCOOH corresponding to about 2% of total sample. Reflectron positive MALDI mass spectra were recorded on a PerSeptive Voyager Elite mass spectrometer (Framingham, Mass., USA) at 20 kV accelerating potential in the delayed extraction mode using standard settings for delay times and grid voltages. Samples were irradiated by a nitrogen laser pulse at 337 nm and 256 laser shots were summed into a single mass spectrum. Spectra were calibrated internally on known background signals.

The peptides were also analyzed by Nano-electrospray Tandem Mass Spectrometry: An aliquot (12 μL out of total of about 40 μL) of the sample solution was concentrated/desalted on a needle which was packed with ˜100 nL of POROS R3 sorbent (PE Biosystems, Framingham Mass., USA). After a washing step with 16 μL of 2% formic acid in aqueous solution, the peptide mixture was directly eluted into a gold-coated borosilicate needle (Protana, Odense, D K) by the addition of 1-2 μL of 2% formic acid in 50/48 v/v CH₃OH/H₂O. The spraying process was started by applying a voltage difference (900 V) between the needle tip and the orifice (1.5 mm distance) of the PE-Sciex QSTAR mass spectrometer (Toronto, Canada). A full mass spectrum was acquired over a wide mass range (m/z 350-1800) and parent ions (mass window of 4.5 Da) of interest were mass-selected and fragmented by collision induced dissociation with nitrogen (collision energy 141 eV; collision gas pressure 6 arbitrary units).

FIG. 2 shows selected mass regions of MALDI mass spectra of tryptic hydrolysates derived from γ-synuclein isoforms. The upper trace corresponds to the Val¹¹⁰ isoform (Spot 438) and the lower trace to the Glu¹¹⁰ isoform (Spot 278). Peptides corresponding to fragments of the different isoforms are labeled accordingly. In FIG. 3 the NanoESMS/MS spectrum of m/z=571.2927 (3+), Mr=1710.8546 Da is consistent with peptide sequence: (98-113)=EDLRPSAPQQEGVASK, derived from the Val¹¹⁰ variant of human γ-synuclein. The difference (99.07 Da) between the masses of fragment ions y₃ (305.17 Da) and y₄ (404.24 Da) confirms the presence of a valine residue in position 110. This is corroborated by the existence of fragment ions (y₅, y₆, y₇, y₈, y₉, y₁₃, y₁₄, and y₁₅), which match this peptide sequence. In FIG. 4 the NanoESMS/MS spectrum of m/z=581.2907 (3+), Mr=1740.8486 Da corresponds to peptide sequence: (98-113)=EDLRPSAPQQEGEASK, derived from wild-type (Glu¹¹⁰) human γ-synuclein. The difference (129.04 Da) between the masses of fragment ions y₃ (305.18 Da) and y₄ (434.22 Da) indicated the presence of a glutamic acid residue in position 110. This is corroborated by the existence of fragment ions (y₅, y₆, y₇, y₈, y₉, y₁₀, y₁₁, y₁₂, y₁₃, y₁₄, and y₁₅) which match this peptide sequence.

TABLE I
Distribution of γ-synuclein isoforms

| | |
|---|---|
| Group 1 (patients) | Group 2 (controls) |

| Sample | 110V | 110E | Sample | 110V | 110E |
|---|---|---|---|---|---|
| 2 | | 110E | 1 | | 110E |
| 3 | 110V | 110E | 4 | | 110E |
| 6 | 110V | 110E | 5 | | 110E |
| 8 | 110V | 110E | 7 | | 110E |
| 9 | 110V | 110E | 10 | | 110E |
| 11 | 110V | 110E | 12 | | 110E |
| 14 | 110V | 110E | 13 | | 110E |
| 16 | 110V | 110E | 15 | 110V | 110E |
| 17 | 110V | 110E | 18 | | 110E |
| 19 | 110V | 110E | 20 | | 110E |
| 22 | | 110E | 21 | | 110E |
| 25 | 110V | 110E | 23 | | 110E |
| 26 | | 110E | 24 | | 110E |
| 27 | 110V | 110E | 29 | | 110E |
| 28 | | 110E | 30 | | 110E |
| 32 | | 110E | 31 | 110V | 110E |
| 35 | 110V | | 33 | | 110E |
| 38 | | 110E | 34 | 110V | 110E |
| 39 | 110V | | 36 | 110V | 110E |
| 40 | | 110E | 37 | | 110E |

We claim:

1. A method of screening for compounds useful for the treatment of SSDs comprising the steps of
a) contacting a γ-synuclein polypeptide with the compounds to be screened;
b) detecting interactions of said compounds with said polypeptide.

2. A method of screening for compounds capable of interfering with the onset of SSDs comprising the steps of
a) contacting a γ-synuclein polypeptide with the compounds to be screened;
b) detecting interactions of said compounds with said polypeptide.

3. The method of claim 1 or 2, wherein the γ-synuclein contains a mutation.

4. The method according to claim 1, 2 or 3 wherein the glutamic acid at position 110 of the γ-synuclein polypeptide is substituted by valine.

5. The method of claim 1, 3, 4 or 4 wherein human γ-synuclein is contacted with the compounds to be screened.

6. The method according to any of the previous claims, wherein a cell-based assay system is used.

7. A method of screening for agonist or antagonist compounds of γ-synuclein comprising the steps of
a) incubating γ-synuclein expressing cells with a candidate compound;
b) assaying for the interference of said compounds with the interaction of γ-synuclein and other cellular proteins.

8. The method of claim 7, wherein the γ-synuclein contains a mutation.

9. The method according to claim 7 or 8, wherein the glutamic acid at position 110 of the γ-synuclein polypeptide is substituted by valine.

10. The method of claim 7, 8 or 9 wherein the human γ-synuclein is used.

11. The method according to any of the previous claims, wherein a fragment, variant or derivative of γ-synuclein is used.

12. A compound, identified by a method according to any of the previous claims, useful for the treatment of SSDs.

13. A method for the production of a pharmaceutical composition comprising identifying a compound by the method of any of claims 1 to 11 and furthermore mixing the compound identified with a pharmaceutical acceptable carrier.

14. Use of a γ-synuclein polypeptide, a fragment, variant or derivative thereof, for the screening of compounds useful for the treatment of SSDs.

15. The use of a γ-synuclein polypeptide, a fragment, variant or derivative thereof, according to claim 14, wherein the said polypeptide contains a mutation.

16. The use of a γ-synuclein polypeptide, a fragment, variant or derivative thereof, according to claim 14, wherein the glutamic acid at position 110 of the γ-synuclein polypeptide is substituted by valine.

17. An oligonucleotide complementary to a part of the γ-synuclein coding sequence useful for the discrimination of a SNP at position 329 of the γ-synuclein coding sequence.

18. An antibody that binds to γ-synuclein polypeptides, or fragments thereof, said polypeptides having a mutation at amino acid position 110 where glutamic acid is substituted for valine, wherein said antibody will bind to an epitope comprising the mutation at position 110 and has a lower binding affinity for the corresponding non-mutated γ-synuclein or fragment thereof.

19. An antibody according to claim 18, said antibody being a monoclonal antibody.

20. Use of a γ-synuclein polypeptide or a nucleotide sequence encoding γ-synuclein for the diagnosis of SSDs or susceptibility to SSDs.

21. A method for the diagnosis of SSDs or susceptibility to SSDs comprising the steps of
a) obtaining cell(s) or a tissue sample from a potential patient;
b) determining the presence or absence of valine at position 110 of a γ-synuclein polypeptide with the antibody of claim 18 or claim 19.

22. A method for the diagnosis of SSDs or susceptibility to SSDs comprising the steps of
a) obtaining cell(s) or a tissue sample from a potential patient;
b) analyzing the nucleotide sequence of a gene or a part of a gene encoding γ-synuclein;
c) comparing the results obtained with a standard sequence.

23. A method according to claim 22, wherein a polynucleotide encoding γ-synuclein or a fragment thereof is amplified by PCR.

24. A method according to claim 22 or 23, wherein the occurrence of a polymorphism at position 329 of the human γ-synuclein gene is analyzed.

25. A method according to any of to claims 22 to 24, wherein the human wild-type γ-synuclein is the standard sequence.

26. A kit useful for diagnosing SSDs or susceptibility to a SSDs according to any claims 21 to 25 comprising at least one of the following components
a) a polynucleotide of the sequence of γ-synuclein, or a fragment thereof, encompassing a sequence containing a SNP;
b) a nucleotide sequence complementary to that of (a);
c) a γ-synuclein polypeptide or a fragment thereof; or
d) an antibody to a γ-synuclein polypeptide or a fragment thereof, capable of detecting a mutation at position 110 of the polypeptide, the mutation preferably being a substitution of glutamic acid by valine.

27. A transgenic animal useful for the study of SSDs or susceptibility to SSDs having a completely or partially deleted γ-synuclein gene.

28. A transgenic animal useful for the study of SSDs or susceptibility to SSDs comprising an exogenous human γ-synuclein gene.

29. The transgenic animal of claim 28, wherein the endogenous γ-synuclein is completely or partially deleted.

30. The transgenic animal of claim 28 or 29 wherein the expression of the human γ-synuclein is controlled by a tissue specific promoter or enhancer.

31. A transgenic animal useful for the study of SSDs, wherein the endogenous γ-synuclein is modified to include the polymorphism corresponding to position 110 in the human sequence.
